(19) United States
(12) Patent Application Publication
(10) Pub. No.: US 2025/0135088 A1
(43) Pub. Date: May 1, 2025

(54) DIAPHRAGM RETAINER RING FOR OPHTHALMIC SURGICAL CASSETTES

(71) Applicant: Alcon Inc., Fribourg (CH)

(72) Inventors: Steven Benjamin ROLL, San Clemente, CA (US); Behzad HASHEMI, Irvine, CA (US); Dienhung Dao PHAN, Westminster, CA (US); Brad Hua CHEN, Mission Viejo, CA (US); Brian William MCDONELL, Irvine, CA (US); Jiansheng ZHOU, Irvine, CA (US); Roderick S. VAN, Long Beach, CA (US); Vincent A. BAXTER, Temecula, CA (US)

(21) Appl. No.: 18/918,671

(22) Filed: Oct. 17, 2024

Related U.S. Application Data
(60) Provisional application No. 63/594,682, filed on Oct. 31, 2023.

(51) Int. Cl. A61M 1/00

(52) U.S. Cl. CPC A61M 1/72; A61M 2205/121

(57) ABSTRACT

Embodiments disclosed herein provide a surgical cassette including pump assemblies with improved contact between a displacement sensor disposed within a surgical console and a diaphragm disposed within the surgical cassette. Each pump assembly includes a diaphragm retainer ring that provides a sufficient creepage and clearance margin, acts as a lead-in for the displacement sensor, assists in preventing misalignment, and provides robustness for the ultrasonic welding process which couples the diaphragm retainer ring to the surgical console. The diaphragm retainer ring is impregnated with a lubricant so to facilitate seating of the displacement sensor with the cassette. A cantilever portion of the diaphragm retainer ring increases a creepage and clearance margin, as well as increases overall part strength in the event of a collision with the displacement sensor and during ultrasonic welding. An insulator is also provided that further increases the creepage and clearance margin.

### CROSS-REFERENCE TO RELATED APPLICATIONS

This application claims the benefit of U.S. Provisional Application Ser. No. 63/594,682 (filed on Oct. 31, 2023), the content of which is incorporated herein by reference in its entirety.

### INTRODUCTION

Ophthalmic surgical procedures are often classified as anterior segment surgical procedures, posterior segment procedures, or combined anterior segment and posterior segment procedures (i.e., “combined procedures”). The anterior segment refers to the front-most region of the eye, and includes the cornea, iris, and lens. Thus, anterior segment surgical procedures typically include surgeries performed on the iris and/or lens, such as cataract surgery. The posterior segment refers to the back-most region of the eye that includes the anterior hyaloid membrane and the optical structures behind it, such as the vitreous humor, the retina, the choroid, and the optic nerve. Posterior segment surgical procedures typically include retinal and vitreoretinal surgeries. In certain cases, a patient may have pathologies of the eye requiring both anterior and posterior procedures; in such cases, a combined procedure may be performed.

During anterior and/or posterior segment surgery, tissue fragments and other materials may be aspirated or suctioned out of the eye through, e.g., a hollow needle or cannula. Also, during the procedure, an irrigating or infusion fluid may be pumped into the eye to maintain an intraocular pressure (IOP) and prevent collapse of the eye. A surgical cassette having one or more peristaltic and/or venturi pumps and one or more valve assemblies may be operably coupled with a fluidics module of a surgical console and used to facilitate the aspiration/suction and irrigation/infusion functionalities described above. In general, the one or more valve assemblies of the surgical cassette are operable to control the application of pressure and vacuum generated by the one or more peristaltic pumps during the surgical procedure.

However, conventional surgical cassettes have a number of significant shortcomings including, for example, providing insufficient creepage or clearance margins between a displacement sensor disposed on the surgical console and a diaphragm disposed within the surgical cassette, providing insufficient lead-in for the displacement sensor which can result in misalignment between the console and the surgical cassette, and the inability to maintain a robust hermetic seal with the surgical console, among others.

Therefore, there is a need for improved surgical cassettes which address at least some of the drawbacks described above. For example, the cantilever portion of the surgical cassette has to be robust enough to not only allow for correct welding parameters which in turn create a hermetic seal with the cassette, but also to prevent cracking or splitting during the welding process.

### SUMMARY

The present disclosure relates generally to ophthalmic surgical cassettes, diaphragm and diaphragm retainer ring assemblies therefor, and methods of use thereof.

In certain embodiments, a surgical cassette is provided for ophthalmic irrigation or aspiration during a surgical procedure, the surgical cassette comprising one or more pump assemblies, wherein each pump assembly comprises a diaphragm retainer ring coupled to a base of the surgical cassette, and a diaphragm coupled to the base and disposed within a cavity defined within the diaphragm retainer ring, wherein the diaphragm retainer ring comprises a cantilever portion disposed over at least a portion of a top surface of the diaphragm.

In certain embodiments, a method is provided for measuring fluid pressure within a surgical cassette for ophthalmic irrigation or aspiration during a surgical procedure, the method comprising coupling the surgical cassette to a surgical console, disposing a displacement sensor of the surgical console adjacent to a diaphragm of the surgical cassette, providing a creepage and clearance margin defined by a cantilever portion of a diaphragm retainer ring coupled to the surgical cassette and disposed between the diaphragm and the displacement sensor, and measuring a change in a location of the diaphragm, the change in the diaphragm location corresponding to a change in the pressure of a fluid within the diaphragm during the surgical procedure. In certain other embodiments, the displacement sensor may induce an eddy current within the top surface of the diaphragm to ascertain its location. In certain other embodiments, an optical sensor, load sensor, or piezo electric based sensors are used in place of a displacement sensor.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of an ophthalmic surgical system that may be used to perform ophthalmic procedures on an eye, according to certain embodiments.

FIG. 1B is an example of subsystems of a console of the ophthalmic surgical system of FIG. 1A, according to certain embodiments.

FIG. 2A is a back side isometric view of an example surgical cassette which may be operably coupled to a console of an ophthalmic surgical system, according to certain embodiments.

FIG. 2B is a back side elevation view of the surgical cassette of FIG. 2A, according to certain embodiments.

FIG. 3 is an exploded perspective view of the surgical cassette of FIGS. 2A and 2B illustrating the components comprising each of the pump assemblies disposed in the surgical cassette, according to certain embodiments.

FIG. 4 is a top down plan view of a diaphragm retainer ring that is disposed within the pump assemblies of the surgical cassette of FIG. 3, according to certain embodiments.

FIG. 5A is a side cross sectional view of the diaphragm retainer ring of FIG. 4, according to certain embodiments.

FIG. 5B is a side cross sectional view of an alternative configuration of the diaphragm retainer ring of FIG. 5A, according to certain embodiments.

FIG. 5C is a side cross sectional view of an alternative configuration of the diaphragm retainer ring of FIG. 5A, according to certain embodiments.

FIG. 5D is a side cross sectional view of an alternative configuration of the diaphragm retainer ring of FIG. 5A, according to certain embodiments.

FIG. 6 is a perspective view of a diaphragm that is disposed within the pump assemblies of the surgical cassette of FIG. 3, according to certain embodiments.

FIG. 7A is a side cross sectional view of a displacement sensor of the console of the ophthalmic surgical system engaged with a pump assembly of the surgical cassette, according to certain embodiments.

FIG. 7B is a magnified cross sectional view of the engagement, and a creepage curve, between the displacement sensor of the console of the ophthalmic surgical system and the pump assembly of the surgical cassette of FIG. 7A, according to certain embodiments.

FIG. 7C is a magnified cross sectional view of the engagement, and a clearance curve, between the displacement sensor of the console of the ophthalmic surgical system and the pump assembly of the surgical cassette of FIG. 7A, according to certain embodiments.

FIG. 8 is a perspective view of a diaphragm comprising an insulator disposed on its top surface, according to certain embodiments.

FIG. 9A is a magnified cross sectional view of the engagement, and a creepage curve, between the displacement sensor of the console of the ophthalmic surgical system and the pump assembly of the surgical cassette comprising the insulator of FIG. 8, according to certain embodiments.

FIG. 9B is a magnified cross sectional view of the engagement, and a clearance curve, between the displacement sensor of the console of the ophthalmic surgical system and the pump assembly of the surgical cassette comprising the insulator of FIG. 8, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

The present disclosure relates generally to ophthalmic surgical cassettes, diaphragms and diaphragm retainer rings therefor, and methods of use thereof.

Certain embodiments disclosed herein provide diaphragm assemblies for a surgical cassette with improved sensing between a displacement sensor disposed within a surgical console and a diaphragm disposed within the surgical cassette. For example, embodiments herein disclose diaphragm assemblies configured to seal a pressure sensitive diaphragm within the surgical cassette via a hermetic seal, while also interacting with a displacement sensor on the surgical console. A diaphragm retainer ring provides a sufficient creepage and clearance margin, acts as a lead-in for the displacement sensor, assists in preventing misalignment, and provides robustness for the ultrasonic welding process which couples the diaphragm retainer ring to the surgical console. Herein, creepage and clearance margin refers to the distance a stray or errant electric current must travel between two conductors. Specifically, creepage refers to the distance the electric current must travel along a surface of an insulating material in order to reach the other conductor, while clearance refers to the distance the electric current must travel through air in order to reach the other conductor.

In certain embodiments, a cantilever portion of the diaphragm retainer ring increases creepage and clearance margin. Features of the cantilever portion contribute to overall part strength in the event of a collision with the displacement sensor and during ultrasonic welding. Specifically, the cantilever portion is configured to route the creepage curve in a way which avoids contact with any additional surfaces of the diaphragm retainer ring, ultimately limiting the tolerances which affect the final creepage and/or clearance distances and margins. The cantilever portion is also chamfered in a way that acts as a lead-in for the displacement sensor disposed on the surgical console in the event of a collision. Finally, the chamfered region is formed in such a way to limit stress concentrations during the ultrasonic welding process, thereby limiting the probability of cracking or deformation of the internal diameter of the diaphragm retainer ring.

Certain embodiments disclosed herein provide that the diaphragm retainer ring is molded from a material that provides rigidity, hardness, low friction, and may include a molded-in lubricant to aid in sensor engagement and accuracy.

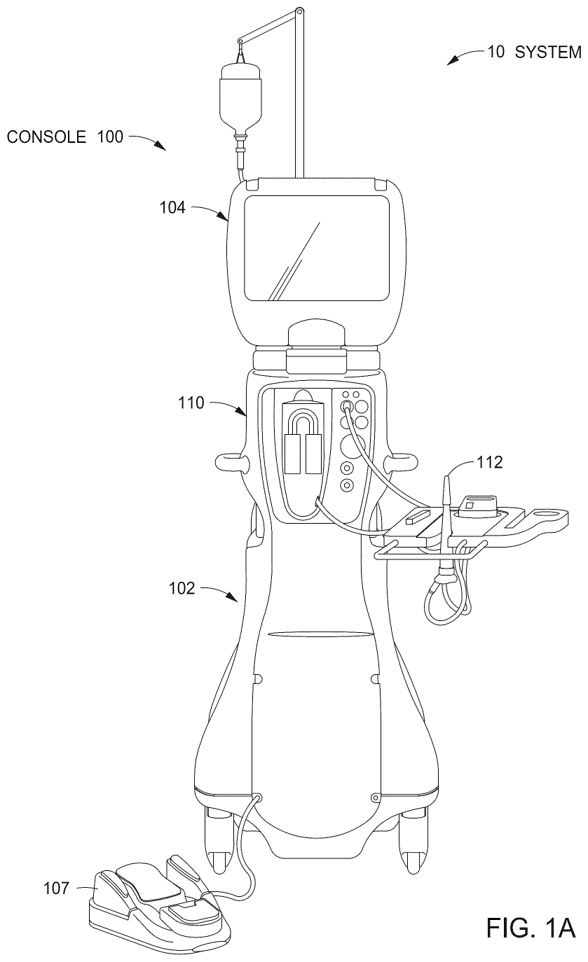

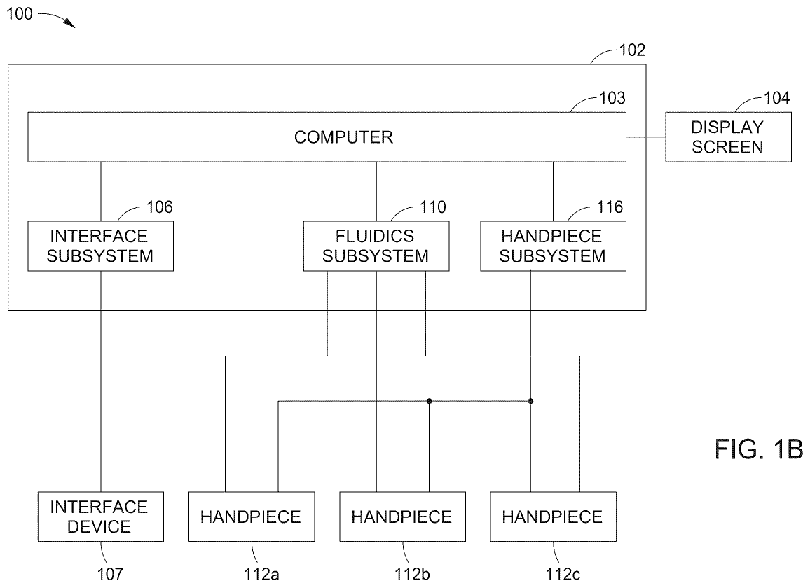

FIG. 1A illustrates an example of an ophthalmic surgical system 10 that may be used to perform ophthalmic procedures on an eye, according to certain embodiments. In the illustrated embodiments, system 10 includes console 100 (also referred to as a “surgical console”), a housing 102, a display screen 104, an interface device 107 (e.g., a foot pedal), a fluidics subsystem 110, and a handpiece 112, coupled as shown and described in more detail with reference to FIG. 1B.

FIG. 1B is an example of subsystems of console 100 of ophthalmic surgical system 10 of FIG. 1A, according to certain embodiments. Console 100 includes housing 102, which accommodates a computer 103 (with an associated display screen 104) and subsystems 106, 110, and 116, which support interface device 107 and handpieces 112 (112a-c). An interface device 107 receives input to surgical system 10, sends output from system 10, and/or processes the input and/or output. Examples of an interface device 107 include a foot pedal, manual input device (e.g., a keyboard), and a display. Interface subsystem 106 receives input from and/or sends output to interface device 107.

Handpiece 112 may be any suitable ophthalmic surgical instrument, e.g., an ultrasonically-driven phacoemulsification (phaco) handpiece, a laser handpiece, an irrigating cannula, a vitrectomy handpiece, or another suitable surgical handpiece. Fluidics subsystem 110 provides fluid control for one or more handpieces 112 (112a-c). For example, fluidics subsystem 110 may manage fluid for an irrigating cannula. Handpiece subsystem 116 supports one or more handpieces 112. For example, handpiece subsystem 116 may manage ultrasonic oscillation for a phaco handpiece, provide laser energy to a laser handpiece, control operation of an irrigating cannula, and/or manage features of a vitrectomy handpiece.

Computer 103 controls operation of ophthalmic surgical system 10. In certain embodiments, computer 103 includes a controller that sends instructions to components of system 10 to control system 10. A display screen 104 shows data provided by computer 103.

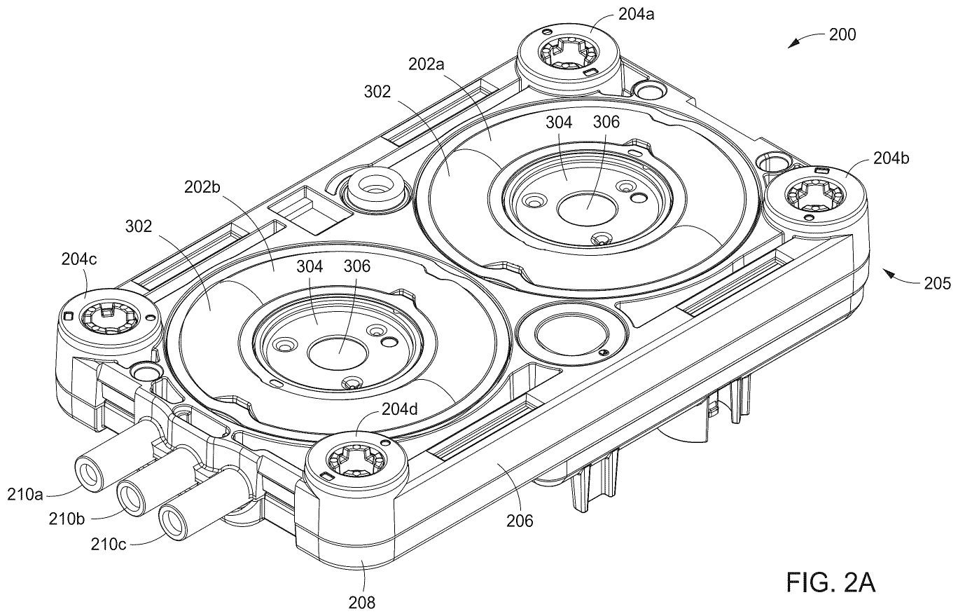

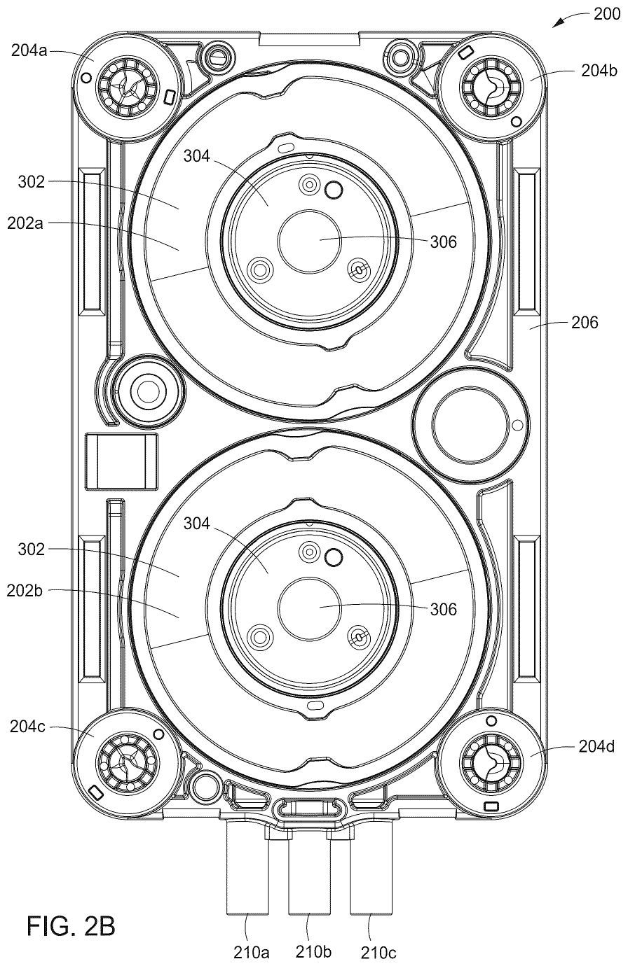

FIG. 2A is a back side isometric view of an example surgical cassette 200 which may be operably coupled to a console of an ophthalmic surgical system (e.g., console 100 of ophthalmic surgical system 10 illustrated in FIGS. 1A-1B), according to certain embodiments. FIG. 2B is a back side elevation view of surgical cassette 200 of FIG. 2A, according to certain embodiments. FIGS. 2A-2B are described together herein for clarity. Surgical cassette 200 includes two pump assemblies 202 (202a-b) which provide a source of pressure and/or vacuum and four valve assemblies 204 (204a-d) which control pressure and/or fluid communication within surgical cassette 200. In certain other embodiments, there may be only one pump assembly or more than two pump assemblies. In certain other embodiments, there may be more or less than four valve assemblies (e.g., two to six valve assemblies).

In certain embodiments, an external source of pressure and/or vacuum is coupled to surgical cassette 200. In such embodiments, the external source may be either in place of or in addition to pump assemblies 202.

Surgical cassette 200 has a housing 205 including a base 206, a cover assembly 208 coupled to base 206, and inlet/outlet ports 210 (210a, 210b, 210c) in base 206 which provide pressure and/or fluid communication between inside and outside of the housing 205. Fluid lines (e.g., tubing) may be coupled between each port 210a-c and a corresponding component of fluidics subsystem 110 and/or a corresponding handpiece 112a-c (shown in FIGS. 1A-1B).

In certain embodiments, one of a first pump assembly 202a or second pump assembly 202b provides a source of pressure (e.g., to create a driving force for fluid irrigation) while the other one of the first pump assembly 202a or second pump assembly 202b provides a source of vacuum (e.g., to create suction for fluid aspiration). The first pump assembly 202a and second pump assembly 202b may be peristaltic pumps or any other suitable type of pump for generating pressure and/or vacuum. In certain embodiments, the first pump assembly 202a and second pump 202b assembly are identical to each other.

Valve assemblies 204 are coupled to base 206. Valve assemblies 204 function cooperatively to control pressure and/or fluid communication within and through surgical cassette 200. In the illustrated embodiments, surgical cassette 200 includes a first valve assembly 204a, a second valve assembly 204b, a third valve assembly 204c, and a fourth valve assembly 204d. As shown, in the embodiments of FIG. 2A, the four valve assemblies 204 are arranged at the four corners of base 206 and surrounding the two pump assemblies 202 which are arranged towards a center of base 206. However, in certain other embodiments, pump assemblies 202 and valve assemblies 204 may have any other suitable arrangement.

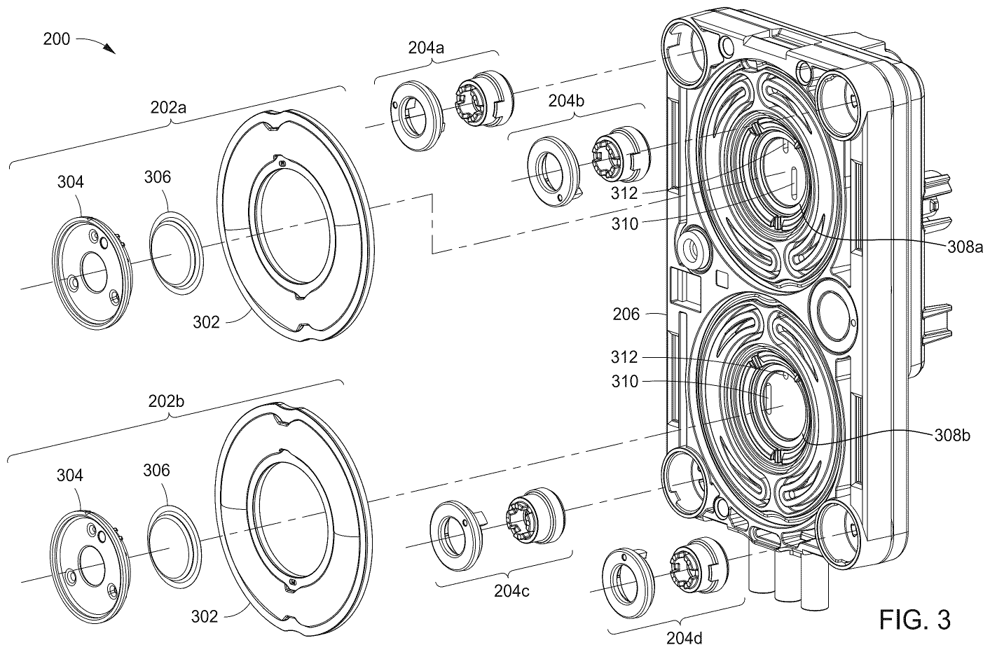

As seen in the exploded view of FIG. 3, each pump assembly 202a, 202b is coupled to and disposed within a first well 308a and a second well 308b, respectively, that are defined within the base 206. Each well 308a, 308b comprises an inlet 310 and an outlet 312 defined therein, the inlet 310 and outlet 312 being in fluid communication with the internal channels disposed within the surgical cassette 200. Each pump assembly 202a, 202b comprises a pump elastomer 302 which is disposed around an outer circumference of each well 308a, 308b. In certain embodiments, each of the first pump assembly 202a and the second pump assembly 202b is configured to engage with one or more rollers of a roller pump disposed on a surgical console, such as console 100. For example, when the surgical cassette 200 is coupled to the console 100, the pump elastomer 302 of each pump assembly 202a, 202b may be contacted against one or more rollers of a roller pump on the console 100. Thereafter, during use, manual forces provided by the rollers rolling along and pressing against the pump elastomers 302, as driven by the pump, may drive fluid within the corresponding pump assembly 202a or 202b, and thus surgical cassette 200, to generate pressure or vacuum as needed. Disposed in a center portion of each well 308a, 308b in a substantially nested or stacked configuration is a diaphragm 306 which is accommodated or disposed below a diaphragm retainer ring 304. In certain embodiments, the diaphragm retainer ring 304 is coupled to the internal surfaces of the well 308a, 308 through an ultrasonic welding process with the diaphragm 306 disposed underneath, the diaphragm retainer ring 304 thereby maintaining the diaphragm 306 within each respective well 308a, 308b and providing a hermetic seal with the base 206.

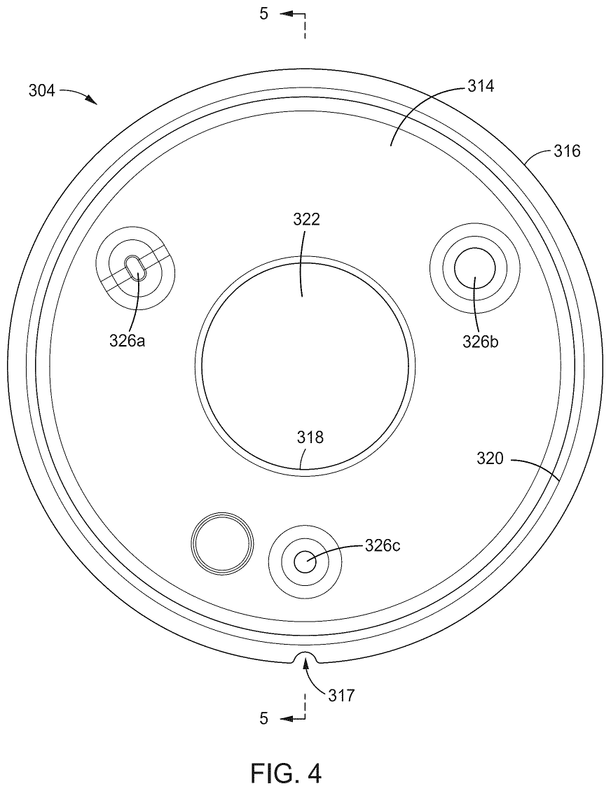

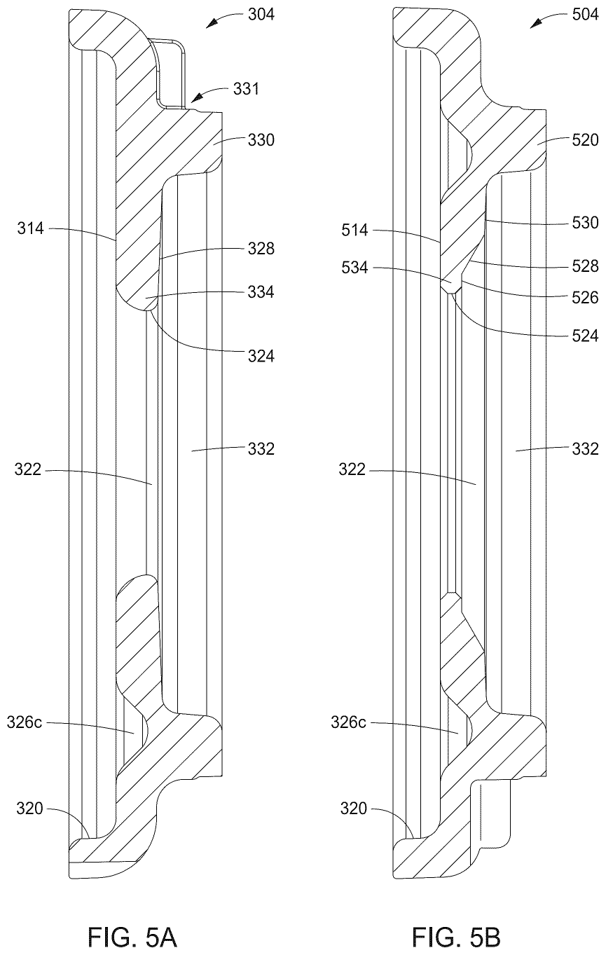

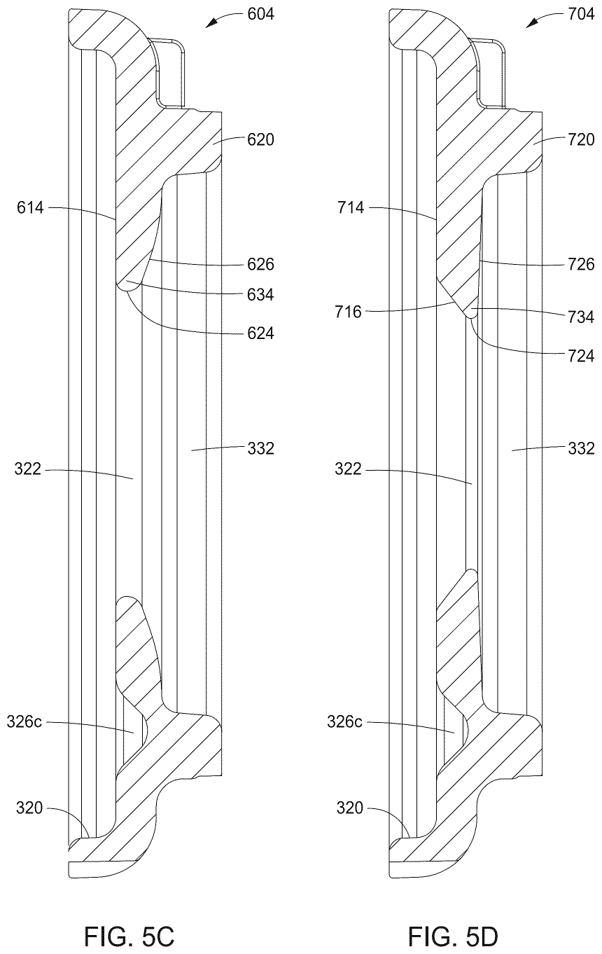

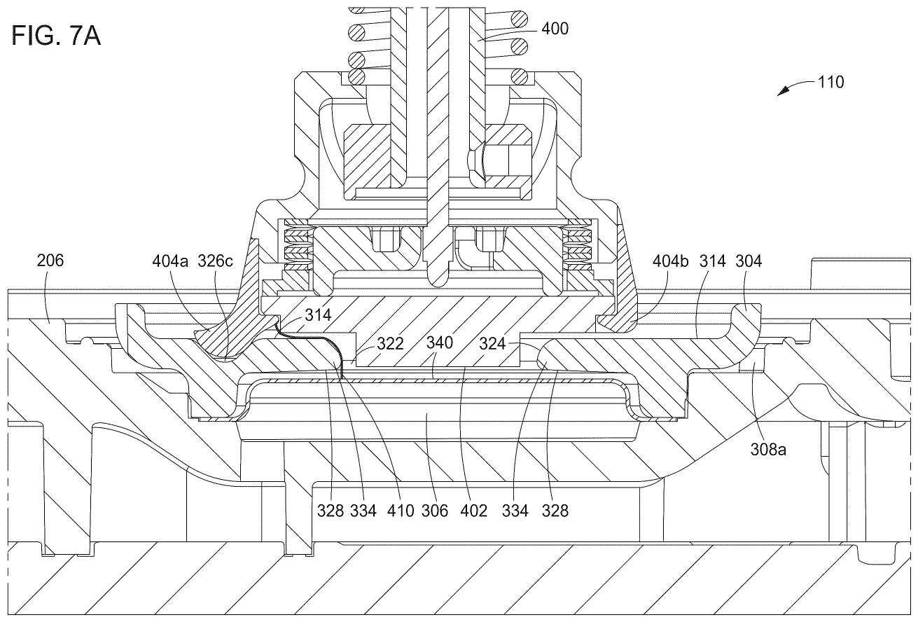

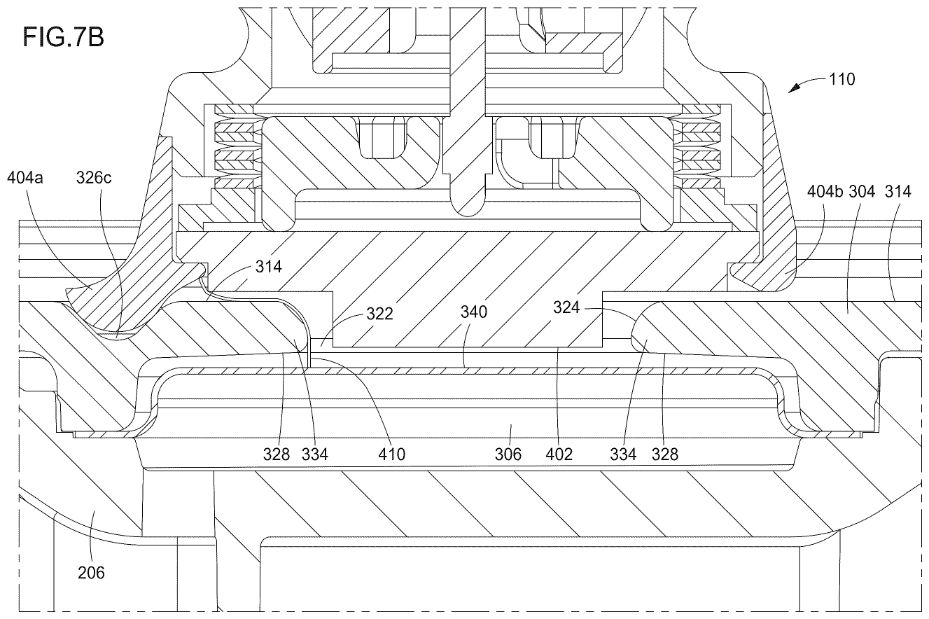

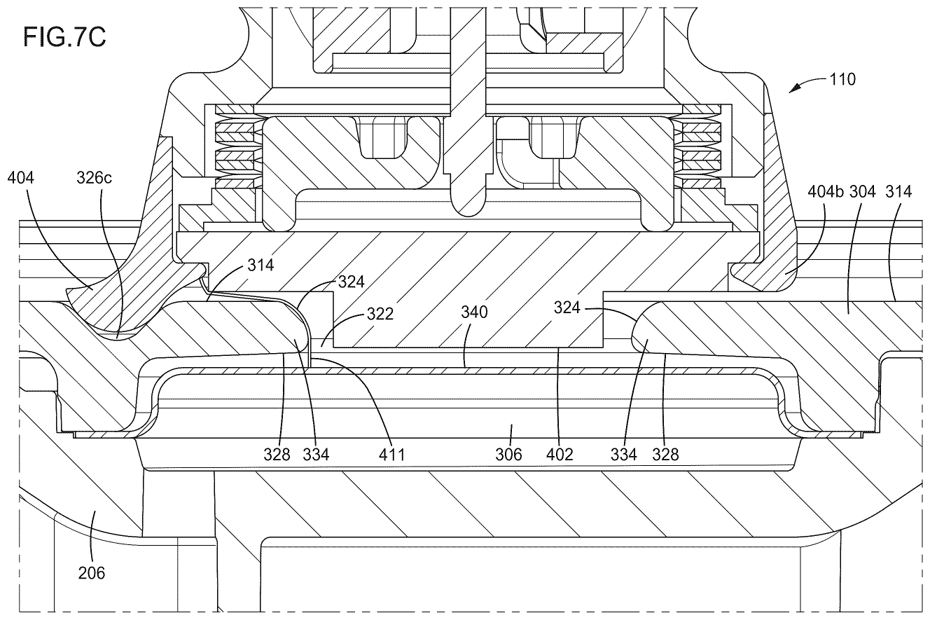

Greater detail of the diaphragm retainer ring 304 is shown, from a top down plan view and a cross-sectional view, respectively, in FIGS. 4 and 5A-5D. The diaphragm retainer ring 304 comprises an annular or ring shaped top surface (or upper surface) 314 defined between an outer circumference 316 and an inner circumference 318. The top surface 314 is circumferentially surrounded by a raised lip or edge 320. Defined in a center of the diaphragm retainer ring 304 and within the inner circumference 318 is an aperture 322, which permits a displacement sensor disposed in the surgical console 100 direct access to a surface of the diaphragm 306 disposed beneath the diaphragm retainer ring 304 as detailed in FIGS. 7A and 7B. Also, defined in the top surface 314 are a plurality of locating features 326 (326a, 326b, 326c). The plurality of locating features 326 are symmetrically defined within the top surface 314. In certain embodiments, each of the locating features 326 comprise a different footprint, shape, cross-sectional depth, profile, or friction coefficient. For example, the first locating feature 326a comprises an angled or V-slot shaped cross sectional profile, while the second locating feature 326b comprises a substantially flat cross sectional profile, and the third locating feature 326c comprises a conical cross sectional profile. In certain embodiments, the raised lip or edge 320 also functions as a lead-in or locating feature for the pressure sensor 400 in the same manner described below for the locating features 326. Also seen in FIG. 4 is a notch 317 that is defined within the outer circumference 316 of the diaphragm retainer ring 304 that may assist in facilitating rotational alignment of the diaphragm retainer ring 304 relative to the base 206 during fabrication. In certain embodiments the notch 317 comprises a substantially semi-circular or half-moon shape; however in other embodiments, other shapes may be used.

In certain embodiments, the diaphragm retainer ring 304 is formed of thermoplastic polymeric material which is resilient enough to withstand impact forces in the event of a collision with the displacement sensor or any other part of the surgical console 100, but yet compatible to bond with and provide a hermetic seal in conjunction with the diaphragm 306 when the diaphragm retainer ring 304 is coupled to the base 206. For example, the diaphragm retainer ring 304 may be formed of a material robust enough to prevent or reduce damage or cracking to an inner diameter of the diaphragm retainer ring 304, which would decrease creepage and/or clearance distance. In further embodiments, the diaphragm retainer ring 304 is formed of a suitable material other than a thermoplastic polymeric material.

In certain embodiments, the material of the diaphragm retainer ring 304 includes or is impregnated with a “molded-in” lubricant so as to provide a desired coefficient of friction across the top surface 314 and within the locating features 326a-c. This facilitates positioning of a displacement sensor when attaching surgical cassette 200 to the surgical console 100, as the displacement sensor of the surgical console 100 may move more easily across the top surface 314 and within locating features 326a-c of the diaphragm retainer ring 304 to its final position above the diaphragm 306. In some embodiments, the diaphragm retainer ring 304 is comprised of up to 10% of lubricant by weight. In some embodiments, the diaphragm retainer ring 304 is comprised of up to 10% or more of lubricant by weight. The lubricant, in certain embodiments, is ultra-high molecular weight silicone or siloxane, however other lubricants may be used instead, or in addition to, what is disclosed herein. Use of “molded-in” lubricant leads to several advantages including a cleaner production process, a reduced likelihood of lubricant making contact with the end user or patient, the prevention of particulate matter from inadvertently attaching to the surface of the diaphragm retainer ring 304 and/or other cassette components, and ensuring that the lubricant is always in its intended location and at the appropriate amount. In certain other embodiments, a lubricant may be applied directly to the top surface 314 and within locating features 326a-c in addition to or instead of the diaphragm retainer ring 304 itself being comprised of a lubricating material.

As shown in FIG. 5A, the inner circumference 318 which defines the aperture 322 comprises a convex or rounded surface or edge 324. The rounded surface 324 begins at the top surface 314 and curves downward until terminating at a bottom surface 328, the bottom surface 328 being substantially parallel relative to the top surface 314. The length of bottom surface 328 is defined between the end of the rounded surface 324 and an annular or ring shaped side wall 330, the bottom surface 328 and the side wall 330 forming the outer boundary of a cavity 332 used to accommodate the diaphragm 306 therein as detailed further below. Also seen in FIG. 5A is an alignment feature 331 that projects outward from the side wall 330 of the diaphragm retainer ring 304. The alignment feature 331 corresponds to a matching or correspondingly shaped feature within the well 308a, 308b so that, when the diaphragm retainer ring 304 is coupled to the surgical cassette 200, the diaphragm retainer ring 304 is correctly aligned relative to the well 308a, 308b. FIG. 5A further illustrates how the top surface 314, rounded surface 324, and bottom surface 328 defined by cavity 332 form a corresponding top, side, and bottom of an annular cantilever portion 334 of the diaphragm retainer ring 304. Beginning from the side wall 330, the cantilever portion 334 extends in a radial direction toward the center of the aperture 322 so as to provide an overhanging or extending portion of the diaphragm retainer ring 304 which covers a corresponding proportion of a top surface 340 of the diaphragm 306. The top surface 314, rounded surface 324, and bottom surface 328 provide the cantilever portion 334 with a configuration and cross-sectional thickness which limits stress concentrations therein which in turn reduces cracking and/or deformation to the cantilever portion 334 when the diaphragm retainer ring 304 is being ultrasonically welded to the surgical cassette 200 during the production process. According to certain embodiments, the cantilever portion 334 further functions as a lead-in or locating feature for the pressure sensor 400 in the same manner described below for the locating features 326.

FIGS. 5B-5D depict a number of different embodiments of the diaphragm retainer ring 304 shown in FIG. 5A. FIG. 5B shows an alternative diaphragm retainer ring 504 comprising a cantilever portion 534 that is defined by a top surface 514 which extends radially inward from a side wall 520. The top surface 514 terminates in a substantially semi-circular shaped or rounded edge 524. A bottom of the cantilever portion 534 is defined by a first bottom surface 526 that is substantially parallel relative to the top surface 514. The first bottom surface 526 leads to an angled surface 528 that in turn leads to a second bottom surface 530. In certain embodiments, both the angled surface 528 and the second bottom surface 530 are disposed at an angle relative to the top surface 514. According to certain embodiments, the angled surface 528 is disposed at a larger angle relative to the top surface 514 than the second bottom surface 530. According to certain embodiments, the cantilever portion 534 further functions as a lead-in or locating feature for the pressure sensor 400 in the same manner described below for the locating features 326.

FIG. 5C shows an alternative diaphragm retainer ring 604 comprising a cantilever portion 634 that is defined by a top surface 614 which extends radially inward from a side wall 620. The top surface 614 terminates in a substantially curved or rounded edge 624. A bottom of the cantilever portion 634 is defined by a bottom surface 626 which leads from the rounded edge 624 to the side wall 620, the bottom surface 626 comprising a substantially convex shape. According to certain embodiments, the cantilever portion 634 further functions as a lead-in or locating feature for the pressure sensor 400 in the same manner described below for the locating features 326.

FIG. 5D shows an alternative diaphragm retainer ring 704 comprising a cantilever portion 734 that is defined by a top surface 714 which extends radially inward from a side wall 720. The top surface 714 terminates at an angled surface 716 which leads to a substantially curved or rounded edge 724, the angled surface 716 comprising a flat or planar configuration. A bottom of the cantilever portion 734 is defined by a bottom surface 726 that is substantially parallel relative to the top surface 714, the bottom surface 726 joining or linking the rounded edge 724 and the side wall 720. According to certain embodiments, the cantilever portion 734 further functions as a lead-in or locating feature for the pressure sensor 400 in the same manner described below for the locating features 326.

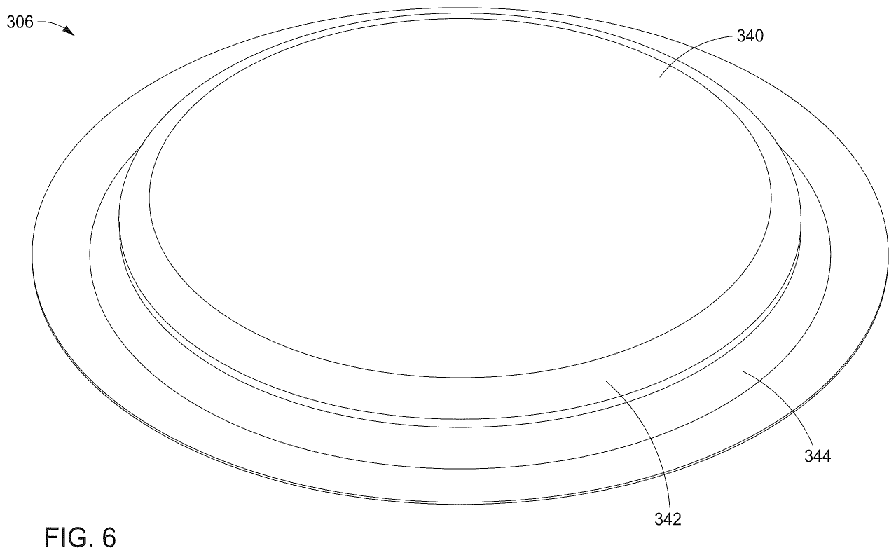

Greater detail of the diaphragm 306 is shown, from a perspective view, in FIG. 6. In certain embodiments, the diaphragm 306 comprises a flat or horizontal top surface 340 that is substantially circular in shape. A side wall 342 surrounding the circumference of the top surface 340 joins the top surface 340 to an annular foot 344 that is disposed about the circumference of the side wall 342. In certain embodiments, the foot 344 is parallel relative to the top surface 340. In certain embodiments, the side wall 342 comprises a curvature, such as a sinusoidal curvature, so as to provide rounded surfaces between the top surface 340 and the foot 344. The diaphragm 306 further comprises a hollow interior so that when it is coupled to the bottom surface of a corresponding well 308a, 308b, fluid entering the well 308a, 308b fills an internal portion of the diaphragm 306 up to an underside of the top surface 340. As fluid exerts pressure on the underside of the top surface 340, the top surface 340 of diaphragm 306 is deflected accordingly which is detected and measured by the displacement sensor disposed in the surgical console 100 detailed further below.

FIGS. 7A and 7B are cross sectional top down views illustrating the engagement between the cassette 200 and the surgical console 100, specifically between a displacement sensor 400 disposed within the fluidics subsystem 110 of the surgical console 100 and each pump assembly 202a, 202b coupled to the base 206, according to certain embodiments.

As the cassette 200 is coupled to the surgical console 100, the diaphragm retainer ring 304 acts as a lead-in for the displacement sensor 400 in the event of a misalignment. According to certain embodiments, the displacement sensor 400 comprises a plurality of lead-in structures 404 (404a-c) which correspond to the plurality of locating features 326 defined in the top surface 314 of the diaphragm retainer ring 304. In certain embodiments, lead-in structures 404a, 404b, and 404c disposed on a proximal end of the displacement sensor 400 share the same geometry and dimensions. In certain other embodiments, however, each of the lead-in structures 404 disposed on a proximal end of the displacement sensor 400 comprises a shape, profile, or cross section which corresponds or matches with one of the plurality of locating features 326 so as to provide a substantially nested or fitted engagement there between. It should be noted that in the illustrated embodiment above, a second and third lead-in structure 404 may be present on the displacement sensor 400 which is configured to correspond and engage with the first locating feature 326a and second locating feature 326b. Additionally, in the event of extreme misalignment, the lead-in structures 404 of the displacement sensor 400 may not engage with the locating features 326 of the retainer ring. In that case, the cantilever portion of the retainer ring will absorb some of the impact from displacement sensor 400 and may guide the displacement sensor into the retainer ring aperture 322.

In certain embodiments, as the displacement sensor 400 is brought into close proximity to the diaphragm retainer ring 304, contact is made between the lead-in structures 404 and the locating features 326 of the diaphragm retainer ring 304. The lead-in structures 404 traverse or slide down the locating features 326 as placement of the cassette 200 is adjusted until being properly seated into the corresponding locating features 326. In certain embodiments, sliding of the lead-in structures 404 into locating features 326 is facilitated by a lubricant which is either part of the material comprising the diaphragm retainer ring 304 or which has been applied to the top surface 314 and locating features 326 directly. The displacement sensor 400 is only properly seated and aligned once each of the lead-in structures 404 has been inserted into or disposed on its respective matching locating feature 326. Once properly seated, a sensor surface 402 of the displacement sensor 400 is simultaneously inserted through the aperture 322 of the diaphragm retainer ring 304 and disposed in close proximity to the top surface 340 of the diaphragm 306.

In FIG. 7B, greater detail of the interaction between the cantilever portion 334, the displacement sensor 400, and the diaphragm 306 may be seen according to certain embodiments. In addition to providing a structural lead-in support for the displacement sensor 400, the cantilever portion 334 of the diaphragm retainer ring 304 also provides a sufficient creepage and clearance margin between the displacement sensor 400 and the diaphragm 306. Creepage and clearance margin refers to the distance a stray or errant electric current must travel between two conductors. Specifically, creepage refers to the distance the electric current must travel along a surface of an insulating material in order to reach the other conductor as seen in FIG. 7B, while clearance refers to the distance the electric current must travel through air in order to reach the other conductor as seen in FIG. 7C. For example, in certain embodiments, an electric current starting from the displacement sensor 400 would have to travel along the creepage curve shown by line 410 that is defined by the size, shape, and relative placement of the cantilever portion 334 of the diaphragm retainer ring 304 in order to reach the conductive diaphragm 306. For example, as seen in FIG. 7B, because of the specific thickness and length of the cantilever portion 334, an errant electric current following the creepage curve 410 must first travel along the top surface 314, across and down the rounded surface 324, and then across the air gap between the bottom surface 328 and the top surface 340 of the diaphragm 306 before making contact with the diaphragm 306 itself. It can be appreciated therefore that variations in the thickness of the cantilever portion 334, the overhanging length of the cantilever portion 334 relative to a diameter of the diaphragm 306, and the height of the cantilever portion 334 relative to a height of the diaphragm 306 would in turn increase or decrease a length of the creepage curve 410 accordingly, according to certain embodiments.

Similarly, in certain embodiments, an electric current starting from the displacement sensor 400 would have to travel along the clearance curve shown by line 411 that is defined by the size, shape, and relative placement of the cantilever portion 334 of the diaphragm retainer ring 304 in order to reach the conductive diaphragm 306. For example, as seen in FIG. 7C, because of the specific thickness and length of the cantilever portion 334, an errant electric current following the clearance curve 411, after passing through an air gap defined between the top surface 314 and a bottom portion of the displacement sensor, must first travel across and down the rounded surface 324, and then across the air gap between the bottom surface 328 and the top surface 340 of the diaphragm 306 before making contact with the diaphragm 306 itself. It can be appreciated therefore that variations in the thickness of the cantilever portion 334, the overhanging length of the cantilever portion 334 relative to a diameter of the diaphragm 306, and the height of the cantilever portion 334 relative to a height of the diaphragm 306 would in turn increase or decrease a length of the clearance curve 411 accordingly, according to certain embodiments.

In certain embodiments, the displacement sensor 400 is an eddy current sensor that is configured to detect changes in the location of the top surface 340 of the diaphragm 306. After being properly seated within the diaphragm retainer ring 304, the sensor surface 402 of the displacement sensor 400 is disposed in close proximity to the top surface 340 of the diaphragm 306. During an ophthalmic procedure, fluid is pumped or drawn through the cassette 200 which at certain points enters each respective well 308a, 308b beneath the internal volume of corresponding diaphragm 306. As more fluid enters the internal volume, pressure within the diaphragm 306 increases which deforms the top surface 340 to form at least a partially rounded or convex surface. In other embodiments, when fluid exits or is vacuumed from the internal volume of the diaphragm 306, the internal pressure decreases which can either smooth or flatten out the top surface 340 of the diaphragm, or in other embodiments, form an indentation or concave surface if fluid continues to exit from the diaphragm 306. In either instance, the location of the top surface 340 of the diaphragm 306 changes accordingly. The eddy current sensor includes a coil located behind the surface 402 energized by a high frequency alternating current. The change in the diaphragm location induces a change in the impedance of the sensor coil. The change in impedance is detected which in turn sends a signal indicative of the displacement and therefore pressure measurement to the computer 103 within the surgical console 100. This pressure can be used by the console to regulate performance or to notify the user of the current fluid pressure within each pump assembly 202a, 202b.

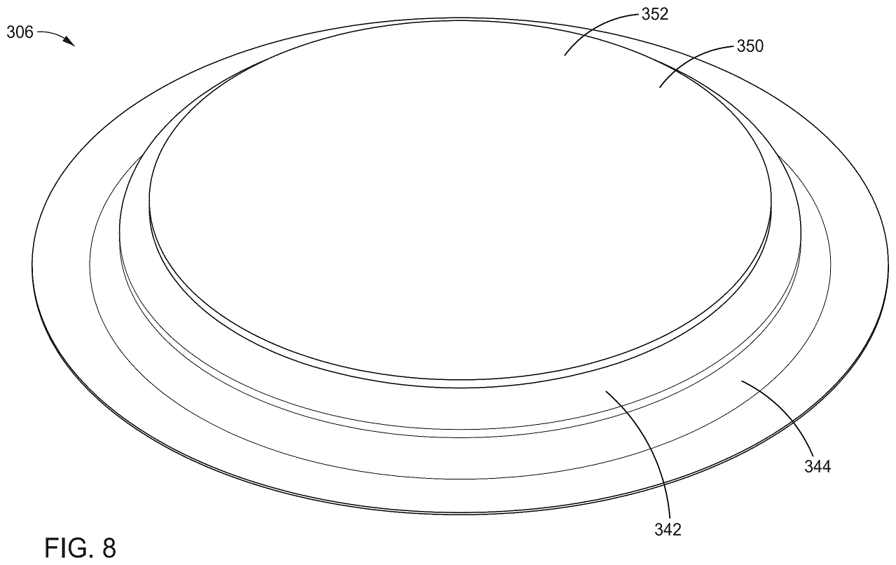

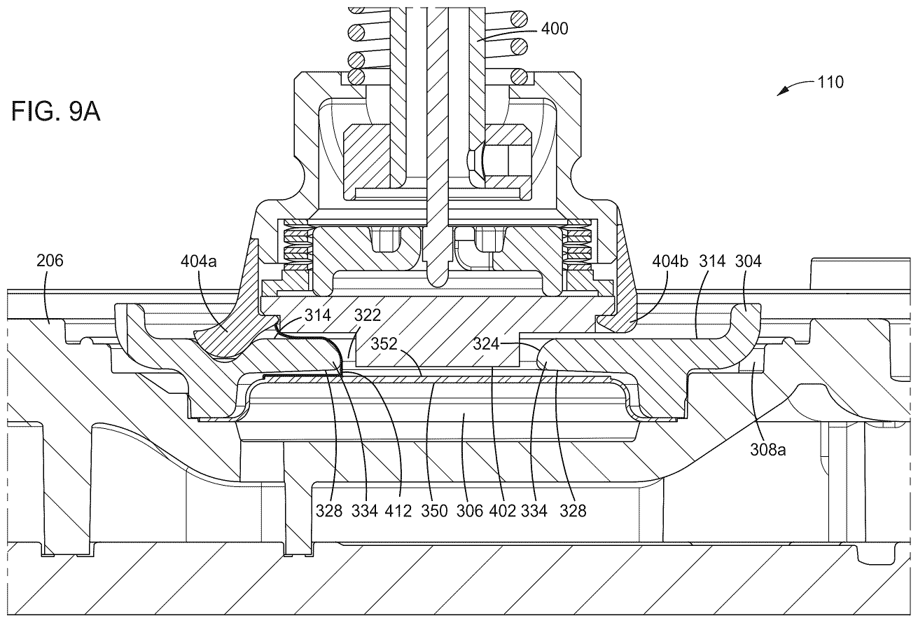

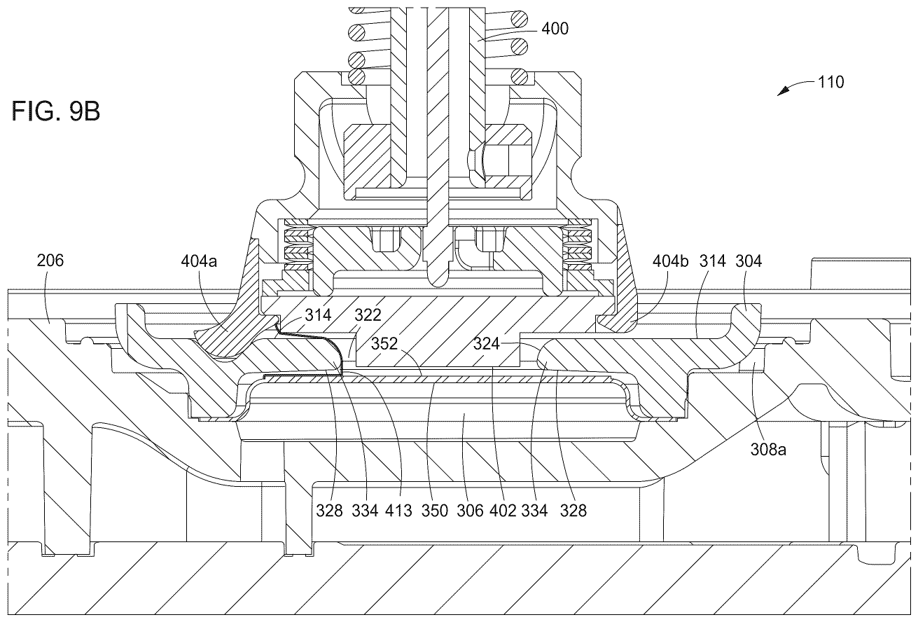

In certain embodiments, an insulator 350 is coupled to or disposed on the top surface 340 of the diaphragm 306 as seen in FIGS. 8 and 9. The insulator 350 comprises an upward facing surface 352 and in certain embodiments is shaped to match the corresponding shape of the top surface 340 of the diaphragm 306. For example, as seen in FIG. 8, the insulator 350 comprises a substantially circular surface area so as to entirely cover the circular top surface 340 of the diaphragm 306. In certain other embodiments, the insulator 350 comprises a diameter that is smaller than the diameter of the diaphragm 306, thereby leaving at least a portion of the top surface 340 of the diaphragm 306 uncovered. In other embodiments, the insulator 350 comprises an annular or ring shape which leaves an inner portion or radius of the top surface 340 of the diaphragm 306 exposed. The insulator 350 is coupled to the diaphragm by an adhesive, however in certain other embodiments, other coupling means such as coating or overmolding are used. In certain embodiments, the insulator 350 is comprised of a layer, or thickness, of electrically insulating material including but not limited to polymers and thermoplastics, for example, polyester films, or polypropylene sheets. However, in other embodiments, additional materials which insulate from electric current but do not affect the induction or measurement of eddy currents such as ceramic or glass can be used.

FIGS. 9A and 9B illustrate how the insulator 350 is incorporated into a pump assembly 202a, 202b, according to certain embodiments. The insulator 350 is coupled to the top surface 340 of the diaphragm 306, with the upward facing surface 352 disposed beneath the bottom surface 328 of the cantilever portion 334 and the sensor surface 402. The insulator 350 in conjunction with the cantilever portion 334 of the diaphragm retainer ring 304 provides or increases a creepage curve seen in FIG. 9A as bold line 412, as well as provides or increases a clearance curve seen in FIG. 9B as bold line 413. In certain embodiments, an errant electric current starting from the displacement sensor 400 must first travel along either the creepage curve 412 or the clearance curve 413 that is defined by the size, shape, and relative placement of the cantilever portion 334 of the diaphragm retainer ring 304, and then across at least a portion of a radial length of the insulator 350, in order to reach the conductive diaphragm 306. For example, as seen in FIG. 9A, because of the specific thickness and length of the cantilever portion 334, an electric current following the creepage curve 412 must travel along the top surface 314, across and down the rounded surface 324, and then across the air gap between the bottom surface 328 of the cantilever portion 334 and the upward facing surface 352 of the insulator 350. The current must then travel across with the width of the upward facing surface 352 and then a height of the insulator 350 before making contact with the diaphragm 306 itself. It can be appreciated therefore that variations in the thickness of the cantilever portion 334, the overhanging length of the cantilever portion 334 relative to a diameter of the diaphragm 306, and the height of the cantilever portion 334 relative to a height of the diaphragm 306, along with variations in the length and height of the insulator 350, would in turn increase or decrease a length of the creepage curve 412 accordingly, according to certain embodiments.

Similarly, as seen in FIG. 9B for example, because of the specific thickness and length of the cantilever portion 334, an electric current following the clearance curve 413, after passing through an air gap defined between the top surface 314 and a bottom portion of the displacement sensor, must travel across and down the rounded surface 324, and then across the air gap between the bottom surface 328 of the cantilever portion 334 and the upward facing surface 352 of the insulator 350. The current must then travel across with the width of the upward facing surface 352 and then a height of the insulator 350 before making contact with the diaphragm 306 itself. It can be appreciated therefore that variations in the thickness of the cantilever portion 334, the overhanging length of the cantilever portion 334 relative to a diameter of the diaphragm 306, and the height of the cantilever portion 334 relative to a height of the diaphragm 306, along with variations in the length and height of the insulator 350, would in turn increase or decrease a length of the clearance curve 413 accordingly, according to certain embodiments.

In certain embodiments, the insulator 350 is coupled to the bottom surface 328 of the cantilever portion 334, to the top surface 314 of the diaphragm retainer ring 304, or at any height that is raised or disposed at a distance relative to the top surface 340 of the diaphragm 306. In other embodiments, the insulator 350 fills a portion of height or space defined between the top surface 340 of the diaphragm 306 and the bottom surface 328 of the cantilever portion 334. In certain other embodiments, the structure of the insulator 350 is incorporated or integral with the structure of the cantilever portion 334 of the diaphragm retainer ring 304 itself.

Accordingly, means for providing sufficient creepage or clearance margins between a displacement sensor disposed on the surgical console and a diaphragm disposed within the surgical cassette, means for providing sufficient lead-in for a displacement sensor, and methods of use thereof are provided herein.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. A surgical cassette for ophthalmic surgical procedures, the surgical cassette comprising:
at least one pump assembly, wherein the at least one pump assembly comprises:
a diaphragm retainer ring coupled to a base of the surgical cassette; and
a diaphragm coupled to the base and disposed within a cavity defined within the diaphragm retainer ring,
wherein the diaphragm retainer ring comprises a cantilever portion disposed over at least a portion of a top surface of the diaphragm.

2. The surgical cassette of claim 1, wherein the diaphragm retainer ring comprises:
a top surface of the diaphragm ring;
a side wall extending downward from the top surface of the diaphragm ring; and
an aperture defined in a center of the top surface of the diaphragm ring,
wherein the cantilever portion is integral with the side wall.

3. The surgical cassette of claim 2, wherein the top surface of the diaphragm ring, the aperture, and the cavity each define an upper surface, a rounded surface, and a bottom surface, respectively, of the cantilever portion of the diaphragm retainer ring.

4. The surgical cassette of claim 3, wherein the bottom surface is at least substantially parallel relative to the top surface of the diaphragm ring.

5. The surgical cassette of claim 3, wherein the bottom surface comprises a first bottom surface and a second bottom surface connected by an angled surface, the angled surface and the second bottom surface disposed at an angle relative to the top surface of the diaphragm ring.

6. The surgical cassette of claim 3, wherein the bottom surface comprises a convex shape.

7. The surgical cassette of claim 3, wherein the diaphragm retainer ring provides creepage and clearance curves defined by the upper surface of the cantilever portion, the rounded surface of the cantilever portion, and a gap defined between the bottom surface of the cantilever portion and the diaphragm.

8. The surgical cassette of claim 2, wherein the cantilever portion extends in a radial direction toward the center of the aperture.

9. The surgical cassette of claim 2, further comprising a plurality of locating features symmetrically defined within the top surface of the diaphragm ring.

10. The surgical cassette of claim 9, wherein the plurality of locating features are each configured to correspond with one of a plurality of lead-in structures disposed on a displacement sensor of a surgical console.

11. The surgical cassette of claim 9, wherein the plurality of locating features comprises at least one of a V-slot shaped cross sectional profile, a flat cross sectional profile, or a conical cross sectional profile.

12. The surgical cassette of claim 1, wherein the diaphragm retainer ring comprises at least one lubricant.

13. The surgical cassette of claim 12, wherein the diaphragm retainer ring is comprised up to 10% of the at least one lubricant by weight.

14. The surgical cassette of claim 1, further comprising an insulator disposed between the diaphragm and a surface of a displacement sensor when the surgical cassette is coupled to a surgical console.

15. The surgical cassette of claim 14, wherein the insulator is coupled to the top surface of the diaphragm.
